(19) United States
(12) Patent Application Publication
(10) Pub. No.: US 2019/0192301 A1
(43) Pub. Date: Jun. 27, 2019

(54) Implant Production Method Using Additive Selective Laser Sintering, and Implant

(71) Applicant: Karl Leibinger Medizintechnik GmbH & Co. KG, Mühlheim (DE)

(72) Inventors: Adem AKSU, Villingen-Schwenningen (DE); Frank REINAUER, Emmingen-Liptingen (DE); Tobias WOLFRAM, Dreieich (DE)

(73) Assignee: Karl Leibinger Medizintechnik GmbH & Co. KG, Mühlheim (DE)

(21) Appl. No.: 16/306,001

(22) PCT Filed: May 8, 2017
(86) PCT No.: PCT/EP2017/060903

§ 371 Date: Nov. 30, 2018

(30) Foreign Application Priority Data
Jun. 7, 2016 (DE) 10 2016 110 500.7

(51) Int. Cl. A61F 2/30; A61F 2/28; A61L 27/56; B22F 3/105

(52) U.S. Cl. CPC A61F 2/3094; A61F 2/2875; A61L 27/56; A61L 27/16; A61F 2002/2889; A61F 2002/30454; A61F 2002/3097; B22F 3/1055

(57) ABSTRACT

The invention relates to a method for producing an implant, wherein particles of the group of ultra-high molecular weight polyethylene (UHMWPE) and/or high-density polyethylene (HDPE) and/or polypropylene (PP) are fused together layer by layer by means of a selective laser sintering method. The invention also relates to an implant produced according to said method.

The invention relates to a method for producing an implant, wherein it is already known to process specific particles, especially UHMWPE particles (ultra-high molecular weight polyethylene particles). UHMWPE in this context is understood to be a purified synthetically pure form of the particles.

For example, U.S. Pat. No. 6,641,617 B1 discloses a radiation-treated medical prosthesis made of UHMWPE. Accordingly, UHMWPE is fused, with substantially no detectable free radicals being present.

EP 1 563 857 A2 furthermore discloses a method for producing abrasion-resistant and oxidation-resistant polyethylene (PE). Accordingly, polyethylene is provided at a temperature below the fusing temperature thereof and then is irradiated so as to obtain cross-linking and to generate sufficient heat as well as to at least partially fuse the polyethylene. After that the polyethylene is cooled.

U.S. Pat. No. 8,142,886 B2 discloses a laser-sintered porous polymer device having a core including a particular amount of inorganic material. The core has at least two further layers, with the inorganic material comprising a mixture of at least two components of the group of metal/metallic alloy, calcium phosphate, stainless steel and glass.

From EP 1 276 436 A1 also an implant for a method of improving the wear resistance and the oxidation resistance of an implant is known, wherein UHMWPE is used and irradiation of the implant is carried out above four Mrad. Further, in that case mixing of an oxidation agent with polyethylene powder is disclosed.

From U.S. 2014/0052264 A1 also a porous implant including a plurality of sintered polymer particles is known, with an antioxidant being present on the surface. Thus, this patent application focuses on a porous implant comprising a plurality of polymer particles which are sintered together at a plurality of contact points so as to form a porous network having pores, wherein the plurality of polymer particles may also contain polyethylene. The antioxidant is disposed on a surface of at least some of the polymer particles and/or in the pores of the porous network.

It is the object of the present invention to make available a faster, lower-cost method which can be carried out more easily and which results in implants that are adapted to be integrated more quickly and more successfully into the tissue of a mammal.

According to the invention, this object is achieved by the fact that, for example, exclusively particles of the group of ultra-high molecular weight polyethylene (UHMWPE) and/or high-density polyethylene (HDPE) and/or polypropylene (PP), especially also mixtures made thereof but being different in type, are fused together layer by layer by means of a selective laser sintering method (SLS method). Also, further particles, acting as fillers for example, may be admixed. Hence, each of UHMWPE, HDPE and PP can be used in pure form only per se or in mixing ratios with two components or in a mixture of all three types of particles. As additives and, resp., admixtures, materials such as for example HAP, CaCO3, Mg, alpha/beta TCP or other polyester materials such as e.g. PDLLA, PLGA, PLA, PGA, chitosan fibers, chitosan particles are suitable.

Especially the components UHMWPE, HDPE and PP have proven themselves for use in the production of implants. Said implants at least partially show desired ingrowing of soft tissue and bone tissue. Even first clinical tests subjected to secrecy are successful, especially with appropriate structuring of the new implants. Here especially good ingrowth is obvious.

Advantageous embodiments are claimed in the subclaims and shall be explained in detail in the following.

It is of advantage when the particles for forming a massive body or a (porous) body including entrapped air/porosities are fused together. A long durability and proper load acceptance are achieved apart from quick ingrowth.

When the body has a complete geometry, for example including undercuts and/or recesses, then even the manufacture of patient-specific individual implants will be possible. Even most complex geometries can be produced which enable versatile use on the human body, for example, especially in the cranial, hand, sternal and foot areas.

It has turned out to be advantageous for human tissue growing into the implant when the particles take a potato-like or sphere-like shape.

In this context, it is desirable when the particles in powder form have a diameter between about 20 μm or about 50 μm and about 300 μm.

The particles present as powder grains should have a diameter between about 40 μm and about 200 μm, preferably 140 μm.

In order to be able to efficiently remove any grains, particles and residual powder components from the raw implant as well as later from the finished implant, it is of advantage when a surface treatment is carried out in the form of a plasma treatment, a snow blasting, a pressurized bombarding with frozen CO2 flakes, such as by means of a supersonic application driven by pressurized air, or a ultrasonic bath.

One advantageous example embodiment is also characterized in that a raw implant is subjected to a heat treatment for increasing the strength.

It is of advantage when the heat treatment follows the surface treatment. Especially when a heat treatment is carried out after the selective laser sintering such that the pores of the implant to be produced remain unsealed or open, the stability is improved and ingrowth will be promoted on a proper level.

In order to obtain especially hygienic products, it is advantageous when a gamma sterilization treatment is carried out preferably at about 25 kGy, for example prior to the surface treatment and/or after the heat treatment. As an alternative, also ethylene oxide (ETO)-, E-beam sterilization- and plasma sterilization methods are suitable.

The invention also relates to a method of an intra-operative modification of an implant produced according to a method according to the invention, namely by means of well-targeted introduction of heat.

Furthermore, the invention also relates to an implant produced in the way according to the invention.

Further, this implant can also be further developed in that it is in the form of a CMF implant (cranio-maxillo-facial implant) for reconstruction of a cartilage and/or bone component for a human body, inter alia of a cranial implant.

The inventor illustrated that, with a pore size of up to 600 μm, there will be rapid ingrowth of blood vessels and connective tissue.

Since nutrient matter supply of vital cells within the implant framework is possible merely over a distance of from about 150 μm to about 200 μm, the neogenesis of blood vessels constitutes a decisive process with respect to successful integration of the implant. The method presented now helps to facilitate ingrowing of soft tissue and bones. This comprehensive vascular ingrowth helps to transport important cells which control infections deeply into the implant. At the same time, ingrowing of soft tissue increases the strength of the implant. Thus, the nutrient matter supply and the strength are improved.

In the present invention, three-dimensional implants are produced by means of selective laser sintering (SLS) out of UHMWPE, HDPE and/or PP. Herein, with defined energy input, the UHMWPE and/or HDPE and/or PP powder particles are fused together locally defined. All three components, only two or only one single component then is/are fused together/in itself (in pure form or in a mixture). By means of the fusing layer by layer according to the invention and subsequent solidifying a three-dimensional implant is formed by superimposing or interconnecting plural individual layers.

Hence short-term production of the implants and adaptation of the implants to the respective/intended/desired anatomic region can be guaranteed.

A production of massive and/or porous, geometrically complex, for example patient-specific, individual implants, but also of standard implants, by means of SLS technology becomes possible.

In particular quick adaptations to individual patients are enabled, especially in situ, ergo at the place of operation.

An increase in strength is achieved by a subsequent heat treatment. A surface treatment is beneficial to the ingrowing behavior, especially when a plasma treatment or a CO2-based technology is employed. The option of subsequent intra-operative modification by heat treatment is provided.

Possible realization of mechanical connecting functions shall be mentioned. For example, a combination with other materials such as synthetic materials, e.g. resorbable synthetic materials, may be implemented. An interconnection/joining, for example in the form of a bridge to another material or in the form of a bridge of a different material can be reasonably realized.

The possibility of integrating fixing options in combination with implant geometries is facilitated.

Laser-sintered porous implants having a total porosity between about 5% and about 90%, based on the empty volume relative to the total volume, are preferred by the users and can be produced by the presented method. Even a total porosity of more than 60% can be easily realized.

It is desired when the pore size is between about 100 μm and about 3,500 μm, especially about 80 μm to about 120 μm, preferably amounts to about 100 μm.

It is also possible that all layers of the implant can be manufactured of UHMWPE and/or HDPE and/or PP.

All layers may be in the form of porous layers. It has turned to be advantageous when the porous laser-sintered implant is used in a defined anatomic region. There may also be obtained an interconnecting pore structure. Well-targeted roughening of the surface to about 5 μm up to about 900 μm is imaginable. The porous laser-sintered implant contains no more residual powder particles prior to use, however. The heat treatment is carried out so that no sealing of the pores will take place. An increase in strength between the interconnecting pore strands is obtained. Surface treatment by means of hot air, infrared emitters and/or thermal deburring and/or explosion deburring will take place. This is resulting in fusing/sealing without any pore sealing. At the same time, oxygen and fuel as well as an optional additive may be ignited at about 3,000° C.

Alternatively, also heat treatment using hot air is feasible. In this context, the use of a hot-air stream at a temperature of from 300° C. to 650° C. proves itself. The temperature on the implant is lower during the treatment, however. The distance observed should be about 10 cm to 30 cm. The heat treatment is carried out for about 5 seconds up to 60 seconds. In doing so, a reduction nozzle having a diameter of 14 mm to 9 mm, or a slot nozzle of 50 mm by 2 mm to 5 mm and, resp., 75 mm by 2 mm to 5 mm, or a flat die is used.

It is of advantage when the implant is hydrophobic and/or hydrophilic. For example, one side may be hydrophobic and the other side may be hydrophilic. The basic material may be hydrophobic, for example. In treatments with low-pressure plasma an optimum structure is obtained. The coating may be applied, for example, in such manner that hydrophilic behavior is provided in a particular area, e.g. only on one side. This helps to achieve quicker ingrowth from this side. The implant may be treated with low-pressure plasma.

Therefore, when the implant basically shows the one, e.g. hydrophobic, property, the other property, for example the hydrophilic property, can be caused by means of a coating. This is also possible vice versa.

Said particles of the group consisting of UHMWPE, HDPE and/or PP can also be used exclusively and/or at least significantly/predominantly. Mixtures exclusively therefrom are especially possible.

1. A method for producing an implant, wherein particles of the group of ultra-high molecular weight polyethylene (UHMWPE) and/or high-density polyethylene (HDPE) and/or polypropylene (PP) are fused together layer by layer by means of a selective laser sintering method, wherein a heat treatment is carried out after the selective laser sintering such that the pores of the implant to be produced remain unsealed or open and/or a heat treatment is carried out after the selective laser sintering such that the pores of the implant to be produced are superficially sealed in total or in partial areas only.

2. The method according to claim 1, wherein the particles are fused together for forming a massive body or a porous body including entrapped air.

3. The method according to claim 2, wherein the body has a complex geometry.

4. The method according to claim 1, wherein the particles have a potato-like or sphere-like shape.

5. The method according to claim 4, wherein the particles in powder form have a diameter between about 20 μm and about 300 μm.

6. The method according to claim 5, wherein the particles present as powder grains have a diameter between about 130 μm and about 150 μm.

7. The method according to claim 1, wherein a surface treatment is carried out in the form of a plasma treatment, a snow blasting, a pressurized bombarding by frozen CO₂ flakes or a ultrasonic bath.

8. (canceled)

9. An implant produced according to the method of claim 1.

10. The implant according to claim 9, wherein the implant is formed as a CMF implant for reconstruction of a cartilage and/or bone component for a human body.

11. An implant produced according to the method of claim 2.

12. The implant according to claim 11, wherein the implant is formed as a CMF implant for reconstruction of a cartilage and/or bone component for a human body

13. An implant produced according to the method of claim 3.

14. The implant according to claim 13, wherein the implant is formed as a CMF implant for reconstruction of a cartilage and/or bone component for a human body

15. An implant produced according to the method of claim 4.

16. The implant according to claim 15, wherein the implant is formed as a CMF implant for reconstruction of a cartilage and/or bone component for a human body

17. An implant produced according to the method of claim 5.

18. The implant according to claim 17, wherein the implant is formed as a CMF implant for reconstruction of a cartilage and/or bone component for a human body

19. An implant produced according to the method of claim 6.

20. An implant produced according to the method of claim 7.

21. The implant according to claim 20, wherein the implant is formed as a CMF implant for reconstruction of a cartilage and/or bone component for a human body
